SEQUENCE LISTING (4 sequences)

SEQ ID NO: 1 - protein, 356 aa, Homo Sapiens
MNGTEGPNFYVPFSNKTGVVRSPFEAPQYYLAEPWQFSMLAAYMFLLIMLGFPINFLTLYVMGYQKKLRSMLNYILLNLAVADLFMVFGGFTTTLYTSLHGYFVFGPTGCNLEGFFATLGGEIALWSLVVLAIERYVVVVHATNSQRPRKLLAIMGVAFTWVMALACAAPPLVGWSRYIPEGMQCSCGIDYYTPHEETNNESFVIYMFVVHFIIPLIVIFFCYGIIISKLSHSKGHQKRKALRMVIIMVIAFLICWLPYAGVAFYIFTHQGSDFGPIFMTIPAFFAKTSAVYNPVIYIMMNKQFRTSAQHALTSVSRGSSLKILSKGKRGGHSSVSTESESSSFHSSTETSQVAPA

SEQ ID NO: 2 - DNA, 1071 bp, Homo Sapiens
atgaacgggaccgagggcccaaacttctacgtgcctttctccaacaagacgggcgtggtgcgcagccccttcgaggccccgcagtactacctggcggagccatggcagttctccatgctggccgcctacatgttcctgctgatcatgcttggcttccccatcaacttcctcacgctgtacgtcatgggttaccagaagaaactgagaagcatgctcaactacatcctgctcaacctggccgtggccgacctcttcatggtcttcgggggcttcaccaccaccctctacacctctctgcacgggtacttcgtctttgggcccacgggctgcaacctggagggcttctttgccaccttgggcggtgaaattgcactgtggtccttggtggtcctggccatcgagcggtacgtggtggtggtccacgccaccaacagtcagaggccaaggaagctgttggccatcatgggcgtcgccttcacctgggtcatggctctggcctgtgccgcgccccccctcgtcggctggtccaggtacatcccggagggcatgcagtgctcgtgcgggattgactactacacgccccacgaggagaccaacaatgagtcgttcgtcatctacatgttcgtggtccacttcatcatccccctgattgtcatattcttctgctacgggattatcatctccaagctgtcacactccaagggccaccagaagcgcaaggccctccgcatggtgatcatcatggtcatcgctttcctaatctgctggctgccctacgctggggtggcgttctacatcttcacccatcagggctctgactttggccccatcttcatgaccatcccggctttctttgccaagacttctgccgtctacaaccccgtcatctacatcatgatgaacaagcagttccggacctctgcccagcacgcactcacctctgtgagcagagggtccagcctcaagatcctctccaaaggaaagcgaggtggacattcatctgtttccactgagtctgagtcttcaagttttcactccagcacagagaccagccaagtggcgcctgcctaa

SEQ ID NO: 3 - protein, 348 aa, Homo Sapiens [sequence omitted]

SEQ ID NO: 4 - protein, 360 aa, Homo Sapiens [sequence omitted]

(19) United States
(12) Patent Application Publication
(10) Pub. No.: US 2014/0128800 A1
(43) Pub. Date: May 8, 2014

(54) PHOTOACTIVATABLE RECEPTORS AND THEIR USES

(71) Applicant: UNIVERSITY OF ROCHESTER, Rochester, NY (US)

(72) Inventor: Minsoo Kim, Pittsford, NY (US)

(73) Assignee: UNIVERSITY OF ROCHESTER, Rochester, NY (US)

(21) Appl. No.: 14/128,136

(22) PCT Filed: Jun. 28, 2012
(86) PCT No.: PCT/US2012/044588

§ 371 Date: Dec. 20, 2013

Related U.S. Application Data
(60) Provisional application No. 61/502,125, filed on Jun. 28, 2011.

(51) Int. Cl. C07K 14/705; A61N 5/06

(52) U.S. Cl. CPC C07K 14/705; A61N 5/062
USPC 604/20; 530/350; 536/23.5; 435/320.1; 435/325; 800/13; 435/173.1; 607/88

(57) ABSTRACT

Provided herein is a chimeric photoactivatable polypeptide comprising an opsin membrane receptor, wherein an intracellular domain of the opsin membrane receptor is replaced with a corresponding intracellular domain of a chemokine receptor, a sphingosine-1-phosphate receptor or an ATP receptor and uses thereof. Further provided are methods of treating cancer, injury of the nervous system, autoimmune disease, and graft rejection comprising administering to the subject a cell that expresses the chimeric photoactivatable polypeptide and exposing the cell to a visible light source.

### BACKGROUND

Chemokines are small cytokine proteins that activate cell adhesion molecules and guide directional cell migration through activation of chemokine receptors. Spatial and temporal regulation of chemokine signals is important for directional cell migration during numerous physiological processes including tissue morphogenesis, inflammation, immune responsiveness, wound healing, and regulation of cell growth and differentiation.

### SUMMARY

Provided herein is a chimeric photoactivatable polypeptide comprising an opsin membrane receptor, wherein an intracellular domain of the opsin membrane receptor is replaced with a corresponding intracellular domain of a chemokine receptor, a sphingosine-1-phosphate receptor or an ATP receptor. Nucleic acids encoding the chimeric polypeptide are also provided. Further provided are cells that express the chimeric polypeptide. Also provided is a method of inducing cell migration comprising exposing a cell that expresses the chimeric photoactivatable polypeptide to a visible light source.

### DESCRIPTION OF THE DRAWINGS

FIG. 1 shows the design for a photoactivatable chemokine receptor (rhodopsin-CXCR4 chimera).

FIG. 2A shows the primary structural alignment of wildtype G protein-coupled receptors rhodopsin (SEQ ID NO:3), CXCR4 (SEQ ID NO: 4) and Rhod-CXCR4 (SEQ ID NO: 1). Highly conserved residues appear in grey. The exchanged intracellular domains are indicated in boxes.

FIG. 2B shows expression of a fluorescently labeled Rhod-CXCR4-chimeric polypeptide in human primary T cells.

FIG. 2C shows Fluor4 Ca²⁺ imaging. Intensity traces of HEK293 cells stably transfected with CXCR4 or transiently transfected with fluorescently labeled Rhod-CXCR4-are provided. Cells were stimulated with CXCL12 or 500 nm light followed by Ca²⁺ ionophore (right panel). For Rhod-CXCR4 expressing cells, Ca²⁺ traces in a positive transfectant (dark grey arrow) and a negative transfectant (light grey arrow) are shown.

FIG. 3A shows a schematic of light-mediated in vivo recruitment of T cells in a mouse model.

FIG. 3B is an example of an optical fiber setting.

FIG. 3C shows the attachment of an LED optical fiber to a mouse ear.

FIG. 4 shows freely moving mice with implanted fiber optics on the ear. The top panel shows that the optical fiber was attached to the mouse ear and the mice were kept in a cage with or without light stimulation. The lower left panel shows the attachment of the LED optical fiber on the mouse ear. The lower right panel shows that mice were kept in the dark with or without light stimulation.

FIG. 5 shows the fold change in the homing index, as determined by [DO.11/(CD4-DO.11)] at day 1 (D1), day 2 (D2), and day 3 (D3). The ear was attached with optical fiber with (light)/without (dark) light activation. The homing index was calculated from ear and spleen.

FIG. 6 shows the establishment of a B16 melanoma tumor on the mouse ear.

FIG. 7a shows a chamber for optical fiber attachment to mouse spinal cord during light stimulation.

FIG. 7b is a schematic showing the implantation of the chamber in mice at the T11-T12 vertebra, just below the dorsal fat pad.

FIG. 7c is a photograph showing the spinal cord damaged through the implanted chamber 144 d after surgery.

FIG. 7d is a photograph of the mouse shown in FIG. 7C, with an implanted chamber.

### DETAILED DESCRIPTION

Described herein are chimeric photoactivatable polypeptides such as, for example, chimeric membrane receptors. As utilized herein, a chimeric polypeptide is a polypeptide comprising at least a portion of a membrane receptor and at least a portion of a different membrane receptor. For example, a chimeric polypeptide can be a polypeptide comprising a G protein coupled receptor wherein at least one intracellular domain of the G protein coupled receptor is replaced with a corresponding intracellular domain of a different G protein coupled receptor. G protein coupled receptors typically comprise three intracellular domains or loops and an intracellular carboxy-terminus. Therefore, provided herein are chimeric photoactivatable polypeptides comprising a G protein coupled receptor wherein one, two, or three intracellular domains are replaced with one, two, or three corresponding intracellular domains of a different G protein coupled receptor. For example, provided are chimeric photoactivatable polypeptides comprising a G protein coupled receptor wherein the intracellular carboxy-terminus is replaced with the corresponding intracellular carboxy-terminus of a different G protein coupled receptor. By replacing one or more intracellular domains and/or the carboxy terminus of a G protein coupled receptor with one or more intracellular domains and/or the carboxy-terminus of a different G protein coupled receptor, the chimeric polypeptide can retain the binding site for a G protein coupled receptor, but effect signaling via the intracellular domains obtained from a different G protein coupled receptor. For example, the intracellular domain(s) of a G protein coupled receptor that normally signals via the Gₜ signaling pathway (for example, an opsin receptor) can be replaced with the intracellular domain(s) of a G protein coupled receptor that normally signals via the Gᵢ signaling pathway (for example, a chemokine receptor) such that when the receptor is photoactivated, the receptor signals via the Gᵢ signaling pathway instead of the Gₜ pathway. Thus, the chimeric polypeptide comprises the photoactivatable properties of the opsin receptor and the signaling properties of the chemokine receptor. The chimeric polypeptides set forth herein respond to an optical stimulus, i.e., light, which triggers the release of a secondary messenger in the cell. Upon stimulation, the signaling properties of the chimeric polypeptides disclosed herein can be assessed by measuring cAMP, cGMP, IP₃, arachadonic acid, intracellular Ca²⁺ release or any other second messenger associated with G protein coupled receptor signaling. Effects downstream of second messenger release can also be measured.

As utilized herein, photoactivatable means that the chimeric polypeptide is activated by light. For example, and not to be limiting, the photoactivatable chimeric polypeptides described herein can be activated at wavelengths from about 450 nm to about 515 nm.

Provided herein is a chimeric photoactivatable polypeptide comprising an opsin membrane receptor, wherein an intracellular domain of the opsin membrane receptor is replaced with a corresponding intracellular domain of a chemokine receptor, a sphingosine-1-phosphate receptor or an ATP receptor. The opsin membrane receptor can be any opsin membrane receptor, now known or identified in the future, that can be photoactivated. The chimeric polypeptide can comprise a full length opsin membrane receptor or a fragment thereof that retains the ability to be photoactivated and has the signaling properties of the chemokine receptor, sphingosine-1-phosphate receptor or ATP receptor upon replacement of the intracellular domain(s). The chimeric photoactivatable polypeptide can further comprise a fluorescent label, for example mCherry, green fluorescent protein, cyan fluorescent protein, and the like for visualization of the chimeric polypeptide.

As mentioned above, one, two or three of the first intracellular domain, the second intracellular domain, the third intracellular domain and the carboxy-terminus of the opsin membrane receptor can be replaced. Opsin receptors include mammalian and non-mammalian opsin receptors. For example, the opsin membrane receptor can be a rhodopsin. Examples of a mammalian rhodopsin polypeptide sequence include, but are not limited to, bovine rhodopsin (for example, the polypeptide sequence set forth under GenBank Accession No. P02699 or GenBank Accession No. NP_{—}001014890 encoded by the nucleotide sequence set forth under GenBank Accession No. NM_{—}001014890.1), human rhodopsin (for example, the polypeptide sequence set forth under GenBank Accession No. NP_{—}000530.1 encoded by the nucleotide sequence provided under GenBank Accession No. NM_{—}000539.3), mouse rhodopsin (for example, the polypeptide sequence set forth under GenBank Accession No. NP_{—}663358.1 encoded by the nucleotide sequence set forth under GenBank Accession No. NM_{—}145383.1), dog rhodopsin (for example, the polypeptide sequence set forth under GenBank Accession No. NP_{—}001008277.1 encoded by the nucleotide sequence set forth under NM_{—}001008276.1) and pig rhodopsin (for example, the polypeptide sequence set forth under GenBank Accession No. NP_{—}999386.1 encoded by the nucleotide sequence set forth under NM_{—}214221.1).

Examples of chemokine receptors are provided in Table 1. For example, the chemokine receptor can be CXCR4, CXCR7, CXCR1, CXCR2, CXCR3, CXCR5, CXCR6, CCR1, CCR2, CCR3, CCR4, CCR5, CCR6, CCR7, CCR8, CCR9, CCR10, CCR11, XCR1 or CS3CR1. The GenBank Accession Nos. for the coding sequences (human mRNA sequences) and the GenBank Accession Nos. for the human protein sequences are also provided. One of skill in the art would know that the nucleotide sequences provided under the GenBank Accession numbers set forth herein are available from the National Center for Biotechnology Information at the National Library of Medicine (http://www.ncbi.nlm.nih.gov/entrez/query.fcgi?db=nucleotide). Similarly, the protein sequences set forth herein are available from the National Center for Biotechnology Information at the National Library of Medicine (http://www.ncbi.nlm.nih.gov/entrez/query.fcgi?db=protein).

TABLE 1

| | | Human GenBank | Human GenBank | |
|---|---|---|---|---|
| | | Accession No. for | Accession No. for | Entrez |
| Receptor | Definition | coding sequence | protein | Gene No. |
| CXCR4 | chemokine (C-X-C | NM_003467.2 | NP_003458.1 | 7852 |
| | motif) receptor 4 | | | |
| CXCR7 | chemokine (C-X-C | NM_020311.2 | NP_064707.1 | 57007 |
| | motif) receptor 7 | | | |
| CXCR1 | chemokine (C-X-C | NM_000634.2 | NP_000625.1 | 3577 |
| | motif) receptor 1 | | | |
| CXCR2 | chemokine (C-X-C | NM_001168298.1 | NP_00116161770.1 | 3579 |
| | motif) receptor 2 | NM_001557.3 | NP_001548.1 | |
| CXCR3 | chemokine (C-X-C | NM_001142797.1 | NP_001136269.1 | 2833 |
| | motif) receptor 3 | NM_001504.1 | NP_001495.1 | |
| CXCR5 | chemokine (C-X-C | NM_001716.3 | NP_001707.1 | 643 |
| | motif) receptor 5 | NM_032966.1 | NP_116743.1 | |
| CXCR6 | chemokine (C-X-C | NM_006564.1 | NP_006555.1 | 10663 |
| | motif) receptor 6 | | | |
| CCR1 | chemokine (C-C | NM_001295.2 | NP_001286.1 | 1230 |
| | motif) receptor 1 | | | |
| CCR2 | chemokine (C-C | NM_001123041.2 | NP_001116513.2 | 729230 |
| | motif) receptor 2 | NM_001123396.1 | NP_001116868.1 | |
| CCR3 | chemokine (C-C | NM_001164680.1 | NP_001158152.1 | 1232 |
| | motif) receptor 3 | NM_001837.3 | NP_001828.1 | |
| | | NM_178328.1 | NP_847898.1 | |
| | | NM_178329.2 | NP_847899.1 | |
| CCR4 | chemokine (C-C | NM_005508.4 | NP_005499.1 | 1233 |
| | motif) receptor 4 | | | |
| CCR5 | chemokine (C-C | NM_000579.3 | NP_000570.1 | 1234 |
| | motif) receptor 5 | NM_001100168.1 | NP_001093638.1 | |
| CCR6 | chemokine (C-C | NM_004367.5 | NP_004358.2 | 1235 |
| | motif) receptor 6 | NM_031409.3 | NP_113597.2 | |
| CCR7 | chemokine (C-C | NM_001838.3 | NP_001829.1 | 1236 |
| | motif) receptor 7 | | | |
| CCR8 | chemokine (C-C | NM_005201.3 | NP_005192.1 | 1237 |
| | motif) receptor 8 | | | |
| CCR9 | chemokine (C-C | NM_006641.3 | NP_006632.2 | 10803 |
| | motif) receptor 9 | NM_031200.2 | NP_112477.1 | |
| CCR10 | chemokine (C-C | NM_016602.2 | NP_057686.2 | 2826 |
| | motif) receptor 10 | | | |
| CCR11 | chemokine (C-C | NM_016557.2 | NP_057641.1 | 51554 |
| | motif) receptor- | NM_178445.1 | NP_848540.1 | |
| | like 1 | | | |
| XCR1 | chemokine (C | NM_001024644.1 | NP_001019815.1 | 2829 |
| | motif) receptor 1 | NM_005283.2 | NP_005274.1 | |
| CX3CR1 | chemokine (C-X3-C | NM_001171171.1 | NP_001164642.1 | 1524 |
| | motif) receptor 1 | NM_001171172.1 | NP_001164643.1 | |
| | | NM_001171174.1 | NP_001164645.1 | |
| | | NM_001337.3 | NP_001328.1 | |

Examples of sphingosine-1-phosphate receptors include, but are not limited to, a sphingosine-1-phosphate receptor 1 (for example, the polypeptide sequence set forth under GenBank Accession No. NP_{—}001391.2 encoded by the nucleotide sequence set forth under GenBank Accession No. NM_{—}001400.4), a sphingosine-1-phosphate receptor 2 (for example, the polypeptide sequence set forth under GenBank Accession No. NP_{—}004221.3 encoded by the nucleotide sequence set forth under GenBank Accession No. NM_{—}004230.3), a sphingosine-1-phosphate receptor 3 (for example, the polypeptide sequence set forth under GenBank Accession No. NP_{—}005217.2 encoded by the nucleotide sequence set forth under GenBank Accession No. NM_{—}005226.2). Examples of ATP receptors include, but are not limited to, a P2Y1 receptor (for example, the polypeptide sequence set forth under GenBank Accession No. NP_{—}002554.1 encoded by the nucleotide sequence set forth under GenBank Accession No. NM_{—}002563.2), or a P2Y2 receptor (for example, the polypeptide sequence set forth under GenBank Accession No. NP_{—}058951.1 encoded by the nucleotide sequence set forth under GenBank Accession No. NM_{—}017255.1).

All of the nucleic acid sequences and protein sequences provided under the GenBank Accession numbers mentioned throughout are hereby incorporated in their entireties by this reference.

Variants of the nucleic acids and polypeptides set forth herein are also contemplated. Variants typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the wild type sequence. Those of skill in the art readily understand how to determine the identity of two polypeptides or nucleic acids. For example, the identity can be calculated after aligning the two sequences so that the identity is at its highest level. These methods allow one of skill in the art to align the intracellular domains of an opsin membrane receptor with the intracellular domains of a chemokine receptor, a sphingosine-1-receptor or an ATP receptor.

Another way of calculating identity can be performed by published algorithms. Optimal alignment of sequences for comparison can be conducted using the algorithm of Smith and Waterman Adv. Appl. Math. 2: 482 (1981), by the alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih gov/blast/bl2seq/bl2.html), or by inspection.

The same types of identity can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 that are herein incorporated by this reference for at least material related to nucleic acid alignment. It is understood that any of the methods typically can be used and that, in certain instances, the results of these various methods may differ, but the skilled artisan understands if identity is found with at least one of these methods, the sequences would be said to have the stated identity.

For example, as used herein, a sequence recited as having a particular percent identity to another sequence refers to sequences that have the recited identity as calculated by any one or more of the calculation methods described above. For example, a first sequence has 80 percent identity, as defined herein, to a second sequence if the first sequence is calculated to have 80 percent identity to the second sequence using the Zuker calculation method even if the first sequence does not have 80 percent identity to the second sequence as calculated by any of the other calculation methods. As yet another example, a first sequence has 80 percent identity, as defined herein, to a second sequence if the first sequence is calculated to have 80 percent identity to the second sequence using each of calculation methods (although, in practice, the different calculation methods will often result in different calculated identity percentages).

Provided herein is a chimeric photoactivatable polypeptide comprising a bovine rhodopsin membrane receptor, wherein an intracellular domain of the opsin membrane receptor is replaced with a corresponding intracellular domain of CXCR4. An example of this polypeptide is provided herein as SEQ ID NO: 1. A nucleic acid that encodes SEQ ID NO: 1 is provided herein as SEQ ID NO: 2. As described in the Examples, SEQ ID NO: 1 is a polypeptide comprising a bovine rhodopsin membrane receptor, wherein the first intracellular domain, the second intracellular domain, the third intracellular domain and the carboxy-terminal domain are replaced with the corresponding first intracellular domain, the corresponding second intracellular domain, the corresponding third intracellular domain and the corresponding carboxy-terminal domain of a CXCR4 chemokine receptor.

The chimeric polypeptides set forth herein can be obtained in numerous ways by those skilled in the art. Based on the methods set forth in the Examples, one of skill in the art would know how to make a polypeptide encoded by a nucleic acid comprising an opsin nucleotide sequence and a chemokine receptor nucleotide sequence. For example, one of skill in the art can align an opsin receptor sequence with a chemokine receptor sequence to identify corresponding intracellular domains as well as the corresponding intracellular carboxyl-terminal domain. Similar techniques can be employed to align an opsin receptor sequence with a sphingosine-1-receptor sequence or an ATP receptor sequence. One of skill in the art can then replace one or more intracellular domains of the opsin membrane receptor with one or more corresponding intracellular domains of the chemokine receptor by utilizing standard mutagenesis techniques to create a chimera. Site-directed mutagenesis techniques, for example, oligonucleotide-directed mutagenesis, can be utilized. In oligonucleotide-directed mutagenesis, an oligonucleotide encoding the desired change(s) in sequence is annealed to one strand of the DNA of interest and serves as a primer for initiation of DNA synthesis. In this manner, the oligonucleotide containing the sequence change is incorporated into the newly synthesized strand. See, for example, Kunkel (1985) Proc. Natl. Acad. Sci. USA 82:488; Kunkel et al. (1987) Meth. Enzymol. 154:367; Lewis and Thompson (1990) Nuc. Acids Res. 18:3439; Bohnsack (1996) Meth. Mol. Biol. 57:1; Deng and Nickoloff (1992) Anal. Biochem. 200:81; and Shimada (1996) Meth. Mol. Biol. 57:157. Other methods are used routinely in the art to modify the sequence of a protein or polypeptide. For example, nucleic acids containing a mutation(s) can be generated using PCR or chemical synthesis, or polypeptides having the desired change in amino acid sequence can be chemically synthesized. See, for example, Bang and Kent (2005) Proc. Natl. Acad. Sci. USA, 102:5014-9 and references therein. Also, well known techniques are available for routinely replacing a region(s) of a G-protein coupled receptor with a region(s) from a different G-protein coupled receptor. See, for example, Geiser et al., “Bacteriorhodopsin chimeras containing the third cytoplasmic loop of bovine rhodopsin activate transducin for GTP/GDP exchange,” Protein Sci. 15(7): 1679-90 (2006); Pal-Ghosh et al. “Chimeric exchange within the bradykinin B2 receptor intracellular face with the prostaglandin EP2 receptor as the donor; importance of the second intracellular loop for cAMP synthesis,” Arch. Biochem. Biophys. 415(1): 54-62 (2004); and Yu et al. “Global chimeric exchanges within the intracellular face of the bradykinin B2 receptor with corresponding angiotension II type Ia receptor regions:generation of fully functional hybrids showing characteristic signaling of the AT1a receptor,” J. Cell Biochem. 85(4): 809-19 (2002).

The chimeric polypeptide can optionally a comprise a linker sequence that links an opsin sequence to non-opsin sequence, for example, a chemokine receptor sequence. The linker sequences can vary in length, and can be, for example, from 1 amino acid to 10 amino acids in length, or greater. Appropriate linker sequences can be determined by one of skill in the art, for example by utilizing LINKER (See Crasto and Feng, “LINKER: a program to generate linker sequences for fusion proteins,” PEDS, 13(5): 309-312 (2000)).

Provided herein is an isolated chimeric polypeptide as set forth herein. By isolated polypeptide is meant a polypeptide that is substantially free from the materials with which a polypeptide is normally associated in nature or in culture. The chimeric polypeptide of the invention can be obtained, for example, by expression of a recombinant nucleic acid encoding the polypeptide (for example, in a cell or in a cell-free translation system), or by chemically synthesizing the polypeptide. Cell membranes comprising a chimeric polypeptide disclosed herein are also be obtained.

Nucleic acids encoding the chimeric polypeptides set forth herein are also provided. Further provided is a vector, comprising a nucleic acid set forth herein. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that can regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region which can serve to facilitate the expression of the inserted nucleic acid. See generally, Sambrook et al., Molecular Cloning: A Laboratory Manual. 2nd Ed., Cold Spring Harbor Laboratory, Cold Spring Harbor, N.Y. (1989). The vector, for example, can be a plasmid. The vectors can contain genes conferring hygromycin resistance, ampicillin resistance, gentamicin resistance, neomycin resistance or other genes or phenotypes suitable for use as selectable markers, or methotrexate resistance for gene amplification.

There are numerous E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters are present, such as the lactose promoter system, a tryptophan (Trp) promoter system, a beta-lactamase promoter system, or a promoter system from phage lambda. Additionally, yeast expression can be used. Provided herein is a nucleic acid encoding a disclosed polypeptide wherein a yeast cell can express the nucleic acid. More specifically, the nucleic acid can be expressed by Pichia pastoris or S. cerevisiae.

Viral vectors comprising the nucleic acids are also provided. For example, the nucleic acids can be in an adenoviral vector, an adeno-associated virus vector, an alphavirus vector, a herpesvirus vector, a lentiviral vector, a retroviral vector or a vaccinia virus vector, to name a few.

The expression vectors described herein can also include nucleic acids encoding a chimeric polypeptide under the control of an inducible promoter such as the tetracycline inducible promoter or a glucocorticoid inducible promoter. The nucleic acids disclosed herein can optionally be under the control of a tissue-specific promoter to promote expression of the nucleic acid in specific cells, tissues or organs. For example, the nucleic acid can be under the control of a promoter that promotes expression in an immune cell, for example, a lymphocyte, a macrophage or a monocyte. Cell specific expression in a B cell, a Tcell, a stem cell, an NK cell, a macrophage, a neutrophil, an eosinophil, a monocyte, a dendrite cell, an endothelial cell, or a keratinocyte is also contemplated. Any regulatable promoter, such as a metallothionein promoter, a heat-shock promoter, and other regulatable promoters, of which many examples are known in the art are also contemplated. Furthermore, a Cre-loxP inducible system can also be used, as well as the Flp recombinase inducible promoter system.

Further provided are vectors containing the nucleic acids encoding the chimeric polypeptides in a host cell suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a COS-7 cell, a HELA cell, an avian cell, a myeloma cell, a Pichia cell, a plant cell or an insect cell. The host cell can also be a B cell, a T cell, a stem cell, an NK cell, a macrophage, a neutrophil, an eosinophil, a monocyte, a dendrite cell, an endothelial cell, or a keratinocyte. A number of other suitable host cell lines have been developed and include myeloma cell lines, fibroblast cell lines, and a variety of tumor cell lines such as melanoma cell lines. Populations of host cells are also provided. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, Lipofectamine™ (Invitrogen, Carlsbad, Calif.), or Lipofectin® (Invitrogen) mediated transfection, electroporation or any method now known or identified in the future can be used for other eukaryotic cellular hosts.

Also provided is an animal comprising a host cell that expresses a chimeric photoactivatable polypeptide as described herein. The animal can be a mammal such as a primate, e.g. a human, or a non-human primate. Non-human primates include marmosets, monkeys, chimpanzees, gorillas, orangutans, and gibbons, to name a few. Domesticated animal, such as cats, dogs, etc., livestock (for example, cattle (cows), horses, pigs, sheep, goats, etc.), laboratory animals (for example, ferret, chinchilla, mouse, rabbit, rat, gerbil, guinea pig, etc.) are also included. Thus, veterinary uses are also provided herein.

Further provided is a transgenic non-human animal, wherein the genome of the animal comprises a nucleic acid encoding a chimeric photoactivatable polypeptide described herein. The nucleic acid can be operably linked to a cell-specific or tissue specific promoter. The transgenic animal can be made by methods known in the art. For the purposes of generating a transgenic animal, screening the transgenic animal for the presence of a transgene and other methodology regarding transgenic animals, please see U.S. Pat. Nos. 6,111,166; 5,859,308; 6,281,408 and 6,376,743, which are incorporated by this reference in their entireties. For example, the transgenic animals can be made by a) injecting a transgene comprising a nucleic acid encoding a chimeric photoactivatable polypeptide linked to an expression sequence into an embryo and b) allowing the embryo to develop into an animal. The method can further comprise crossing the animal with a second animal to produce a third animal (progeny). Cells comprising a transgene, wherein the transgene comprises a nucleic acid encoding a chimeric photoactivatable polypeptide can be isolated from the transgenic animal. The transgenic animal includes, but is not limited to, mouse, rat, rabbit or guinea pig.

In the transgenic animals described herein, the transgene can be expressed in a specific cell type, for example, a B cell or a T cell. Therefore, a T cell specific expression sequence can be selected such that expression of the transgene is primarily directed to T cells, but not exclusively to T cells. The expression sequence can be, for example, a T cell specific promoter. This example is not meant to be limiting as one of skill in the art would know how to select cell-specific expression sequences to direct expression of the transgene to a particular cell type, for example, a B cell, a stem cell, an NK cell, a macrophage, a neutrophil, an eosinophil, a monocyte, a dendrite cell, an endothelial cell, or a keratinocyte, to name a few.

In the transgenic animal disclosed herein, expression of the transgene can be controlled by an inducible promoter. The transgenic animal of this invention can utilize an inducible expression system such as the cre-lox, metallothionine, or tetracycline-regulated transactivator system. An example of the cre-lox system for inducible gene expression in transgenic mice was published by R. Kuhn et al., “Inducible gene targeting in mice,” Science, 269(5229): 1427-1429, (1995) which is incorporated in its entirety by this reference. Use of the tetracycline inducible system is exemplified in D. Y. Ho et al., “Inducible gene expression from defective herpes simplex virus vectors using the tetracycline-responsive promoter system,” Brain Res. Mol. Brain. Res. 41(1-2): 200-209, Sep. 5, 1996; Y. Yoshida et al., “VSV-G-pseudotyped retroviral packaging through adenovirus-mediated inducible gene expression,” Biochem. Biophys. Res. Commun. 232(2): 379-382, Mar. 17, 1997; A. Hoffman et al., “Rapid retroviral delivery of tetracycline-inducible genes in a single autoregulatory cassette,” PNAS, 93(11): 5185-5190, May 28, 1996; and B. Massie et al., “Inducible overexpression of a toxic protein by an adenovirus vector with a tetracycline-regulatable expression cassette,” J. Virol. 72(3): 2289-2296, March 1998, all of which are incorporated herein in their entireties by this reference.

Also provided is a method of inducing cell migration comprising exposing a cell that expresses a chimeric photoactivatable polypeptide that comprises an opsin membrane receptor, wherein an intracellular domain of the opsin membrane receptor is replaced with a corresponding intracellular domain of a chemokine receptor, a sphingosine-1-phosphate receptor or an ATP receptor to a visible light source. The cells can be in vitro, ex vivo, or in vivo. The visible light source can be any source that emits light in the visible light spectrum, for example, a laser, an optical fiber or a light emitting diode. In the methods set forth herein, cell migration can be induced by exposing the cells to a visible light source that emits light, for example, at a wavelength of about 450 to 515 nm. Methods for assessing light-mediated directional migration of cells in vitro and in vivo are described in the Examples. The cells can be exposed to a timed pulse(s) of light, for example, a pulse(s) of about 15 seconds, 20 seconds, 25 seconds, 30 seconds, 35 seconds 40 seconds or any amount of time in between. The cells can also be continuously exposed to the light source, for example, for about 1 minute, 2 minutes, 3 minutes, 4 minutes, 5 minutes, 10 minutes, 15 minutes, 20 minutes, 25 minutes, 30 minutes, 35 minutes, 40 minutes or any amount of time in between. If timed pulses are employed, one of skill in the art can determine how long each pulse should be and how long the interval between pulses should be. One of skill in the art can also determine whether single or multiple exposures to light are necessary. Exposure times and wavelengths can be determined empirically by exposing the cells to the visible light source, assessing cell migration and adjusting the exposure time, number of pulses, and/or wavelength accordingly.

Further provided is a method of treating cancer in a subject comprising administering to the subject a cell that expresses a chimeric photoactivatable polypeptide that comprises an opsin membrane receptor, wherein an intracellular domain of the opsin membrane receptor is replaced with a corresponding intracellular domain of a chemokine receptor, a sphingosine-1-phosphate receptor or an ATP receptor, and exposing the cell in the subject to a visible light source, wherein the subject has cancer. 10³-10⁸ cells can be administered, including 10³-10⁵, 10⁵-10⁸, 10⁴-10⁷ cells or any amount in between in total for an adult subject This method can optionally comprise the step of diagnosing a subject with cancer.

As used throughout, by subject is meant an individual. Preferably, the subject is a mammal such as a primate, and, more preferably, a human. Non-human primates are subjects as well. Thus, veterinary uses and medical formulations are contemplated herein.

Throughout this application, by treating is meant a method of reducing or delaying one or more effects or symptoms of a disease. Treatment can also refer to a method of reducing the underlying pathology rather than just the symptoms. The treatment can be any reduction and can be, but is not limited to, the complete ablation of the disease or the symptoms of the disease. Treatment can include the complete amelioration of a disease as detected by art-known techniques. For example, a disclosed method is considered to be a treatment if there is about a 10% reduction in one or more symptoms of the disease in a subject when compared to the subject prior to treatment or control subjects. Thus, the reduction can be about a 10, 20, 30, 40, 50, 60, 70, 80, 90, 100%, or any amount of reduction in between.

Cancers that can be treated by the methods set forth herein include, but are not limited to, skin cancer, colon cancer, brain cancer, breast cancer, prostate cancer, esophageal cancer, rectal cancer, throat cancer, lung cancer, eye cancer (for example, retinoblastoma or intraocular cancer, blood cancer (for example, leukemia, lymphoma or myeloma) and stomach cancer.

The cell can be a T cell, a stem cell or an NK cell. For example, and not to be limiting, in tumor immunology, where adoptive cell transfer can be used for anticancer immunotherapy, the therapeutic efficiency of in vitro activated autologous T cells is dependent upon access of the T cells to the tumor sites once they are transferred to patients. A photoactivatable chemokine receptor can guide autologous T cells to the location of a tumor using non-invasive light stimulation to induce directional migration. For example, the T cell(s) can be removed from the subject and transfected ex vivo with a nucleic acid encoding the chimeric photoactivatable polypeptide, prior to administering the cell to the subject. After the cell(s) is administered to the subject, the cell is exposed to a visible light source to induce cell migration to the tumor site. As set forth above, the visible light source can be a laser, an optical fiber or a light emitting diode. If the subject has skin cancer, the cells can be delivered to the subject, for example, by local injection or transdermally, prior to exposing the target of the subject's skin to the visible light source. The cells can also be delivered to a subject intrarectally, for example to treat colon or rectal cancer; intractracheally/intrabronchially, for example to treat lung cancer; laproscopically, for example, to treat liver, pancreatic, or kidney cancer; or intravaginally, for example, to treat cervical or uterine cancer, followed by exposure of the cells to a visible light source via endoscopic methods. In the methods set forth herein, the cells can also be administered to the subject at a surgical site followed by exposure to visible light, for example, via laser or endoscopic methods. Cannulation can also be utilized to insert an optical fiber at a desired site.

The methods of treating cancer can optionally comprise administration of another anti-cancer therapy, for example, surgery, radiation therapy or chemotherapy. Examples of chemotherapeutic agents include, but are not limited to, cisplatin, oxaliplatin, cyclophosphamide, Procarbazine, taxanes, Etoposide, to name a few. Optional anti-cancer treatments can be administered prior to, concurrently with or subsequent to administration of the cells.

Also provided herein is a method of treating a neural injury (e.g., spinal cord injury, stroke, head injury, or peripheral nerve injury) in a subject comprising transplanting a neural stem cell (e.g., a stem cell capable of giving rise to neurons, glial cells (e.g. oligodendrocytes) or both) that expresses a chimeric photoactivatable polypeptide into the spinal cord, brain or nerve of a subject and exposing the cell in the subject to a visible light source, wherein the subject has a spinal cord injury, head injury or peripheral nerve injury. Neural stem cells include pluripotent or totipotent stem cells. Such stem cells can be derived from the same subject, or a different subject, including an embryonic subject. Alternatively, the cells can be induced pluripotent stem cells or induced totipotent stem cells.

Further provided are methods of treating diseases of the central nervous system or peripheral nervous system marked by a loss of neurons or by demyelination. Such diseases include amyotrophic lateral sclerosis (ALS), Parkinsons's disease, multiple sclerosis (MS), Alzheimer's disease, and the like.

The number of stem cells to be administered depends on the type of cell; species, age, or weight of the subject; and the extent or type of the injury or disease. Optionally, administered doses range from about 10³-10⁸, including 10³-10⁵, 10⁵-10⁸, 10⁴-10⁷, cells or any amount in between in total for an adult subject. Cells can generally be administered at concentrations of about 5-50,000 cells/microliter. Optionally, administration can occur in volumes up to about 15 microliters per injection site. However, administration to the central nervous system can involve much larger volumes. The method can further comprise administering a therapeutic agent, for example, an agent utilized to treat spinal cord injury or CNS lesions. For example, several agents have been applied to acute spinal cord injury (SCI) management and CNS lesions that can be used in combination with stem cell transplantation. Such agents include agents that reduce edema and/or the inflammatory response. Exemplary agents include, but are not limited to, steroids, such as methylprednisolone; inhibitors of lipid peroxidation, such astirilazad mesylate (lazaroid); and antioxidants, such as cyclosporin A, EPC-K1, melatonin and high-dose naloxone. These agents can be administered prior to administration of the stem cells, concurrently with the stem cells or subsequent to administration of the stem cells. Thus, the compositions including stem cells can further comprise methylprednisolone, tirilazad mesylate, cyclosporin A, EPC-K1, melatonin, or high-dose naloxone or any combination thereof. Other therapeutic agents that could be administered prior to, concurrently with or after stem cells include tissue plasminogen activator, prolactin, progesterone, growth factors, etc.

Further provided is a method of treating an autoimmune disorder or preventing transplant rejection by administering a regulatory T cell that expresses a chimeric photoactivatable polypeptide to a subject and exposing the cell in the subject to a visible light source, wherein the subject has an autoimmune disorder or has received an organ transplant. The autoimmune disorder can be, but is not limited to, spontaneous type 1 diabetes, psoriasis or arthritis. For subjects that have received an organ or cell transplant, the transplant can be a liver transplant, a kidney transplant, a heart transplant, a lung transplant, a pancreas transplant, a pancreatic islet cells transplant, an intestinal transplant or any of a variety of other transplants. The method can further comprise administering an immunosuppressant, either prior to administration of the regulatory T cells, concurrently with the regulatory T cells or subsequent to administration of the regulatory T cells.

Also provided is a method of treating an infection in a subject comprising administering to the subject a cell that expresses a chimeric photoactivatable polypeptide that comprises an opsin membrane receptor, wherein an intracellular domain of the opsin membrane receptor is replaced with a corresponding intracellular domain of a chemokine receptor, a sphingosine-1-phosphate receptor or an ATP receptor, and exposing the cell in the subject to a visible light source, wherein the subject has an infection. The cell can be an immune cell, for example, a regulatory T cell. The infection can be a parasitic infection, a viral infection, a bacterial infection or a fungal infection.

The cells comprising the chimeric photoactivatable polypeptides set forth herein can be prepared by making a cell suspension of the cultured cells in a culture medium or a pharmaceutically acceptable carrier. Thus, provided herein is a pharmaceutical composition comprising an effective amount of the cells in a pharmaceutically acceptable carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, dextrose, and water.

An agent or agents delivered in combination with the cells can be administered in vitro or in vivo in a pharmaceutically acceptable carrier. A pharmaceutically acceptable carrier for the agent can be a solid, semi-solid, or liquid material that can act as a vehicle, carrier or medium. Thus, compositions can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. A pharmaceutical composition additionally can include, without limitation, lubricating agents such as talc, magnesium stearate, and mineral oil; wetting agents; emulsifying and suspending agents; preserving agents such as methyl- and propylhydroxy-benzoates; sweetening agents; and flavoring agents. Pharmaceutical compositions can be formulated to provide quick, sustained or delayed release after administration by employing procedures known in the art. In addition to the representative formulations described below, other suitable formulations for use in a pharmaceutical composition can be found in Remington: The Science and Practice of Pharmacy (21th ed.) ed. David B. Troy, Lippincott Williams & Wilkins, 2005.

Liquid formulations for oral administration or for injection generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. Compositions for inhalation include solutions and suspensions in pharmaceutically acceptable, aqueous or organic solvents, or mixtures thereof, and powders. These liquid or solid compositions may contain suitable pharmaceutically acceptable excipients as described herein. Such compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent or intermittent positive pressure breathing machine. Solution, suspension, or powder compositions may be administered, orally or nasally, from devices which deliver the formulation in an appropriate manner. Another formulation that is optionally employed in the methods of the present disclosure includes transdermal delivery devices (e.g., patches). Such transdermal patches may be used to provide continuous or discontinuous infusion of an agent described herein.

According to the methods taught herein, the subject is administered an effective amount of the cells. The terms effective amount and effective dosage are used interchangeably. The term effective amount is defined as any amount necessary to produce a desired physiologic response. Effective amounts and schedules for administering the cells can be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Instructions for use of the composition can also be included.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. A number of embodiments have been described. Nevertheless, it will be understood that various modifications may be made. Accordingly, other embodiments are within the scope of the following claims.

### Examples

Chemokines are small cytokine proteins that activate cell adhesion molecules and guide directional cell migration through activation of their cognate receptors. Spatial and temporal regulation of chemokine signals is important for directional cell migration during tissue morphogenesis, inflammation, immune responsiveness, wound healing, and regulation of cell growth and differentiation. The role of chemokine-mediated cell migration in the immune system is particularly complex as immune cell migration regulates many aspects of the immune response. This is because, unlike cells within solid tissues, circulating leukocytes relocate during the course of immune reactions and in so doing dynamically interact with cells of the vasculature and with other immune cells, as well as with components of the extracellular matrix. Insufficient chemokine activity contributes to recurrent infections and impaired wound healing, and excessive chemokine activity leads to an exaggerated inflammatory response and associated tissue damage leading to autoimmune diseases such as rheumatoid arthritis, asthma, diabetes, inflammatory bowel disease and multiple sclerosis, to name a few.

However, major challenges in studying cell migration by specific chemokine signals exist. For example, it remains difficult to manipulate chemokine activity at precise times and places within living animals. Also, it is not possible to study different chemokine effects on defined cell types over a range of timescales. Further, it is difficult to study pulsatile vs. tonic chemokine signals. In addition, a given chemokine can activate multiple chemokine receptors and vice versa. The standard genetic perturbation techniques, such as knockdown, overexpression and mutation are slow in timescale and broad in effect. Injection of pharmacological reagents or surgical perturbations is more likely to destroy and induce a local immune response rather than modulate specific spatiotemporal features of the response and may mask the actual effects of reagents themselves. Therefore, despite recent advances in chemokine research, there are few ways to assess leukocyte behavior in a rapid and specific manner. A light-mediated approach is provided herein because light can be delivered to small, defined areas in timed pulses.

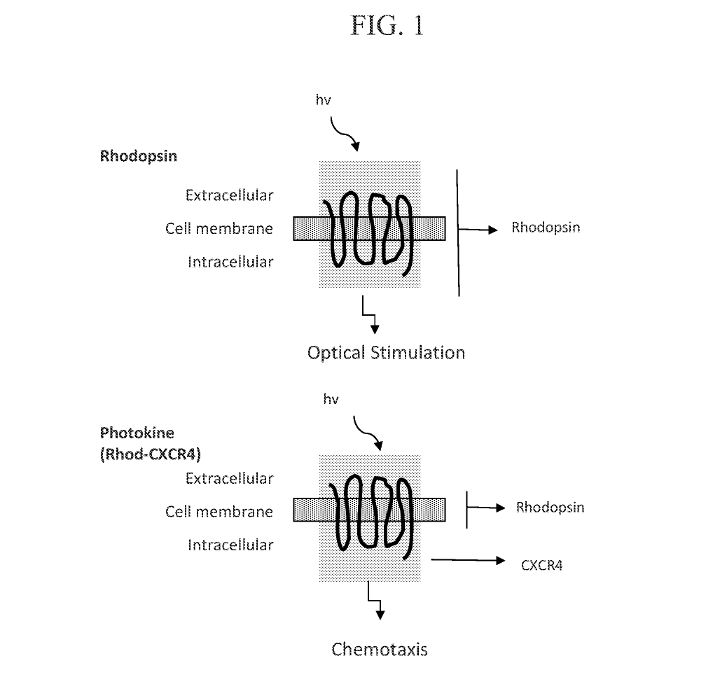

Photoactivatable chemokine receptors were developed that leverage common structure-function relationships between two different GPCR families (a rhodopsin receptor and a chemokine receptor). An example of a photoactivatable receptor is a Rhodopsin-CXCR4 chimera that can regulate cell migration and recruit distinct T cell populations in vivo by inducing migration signals in response to light (FIG. 1). The use of light to control immune reactions avoids the need for direct physical contact with the tissue, and therefore, any interference with normal functions. Importantly, light offers numerous other advantages, such as, for example, outstanding spatial resolution and resolution of signals in all types of lymphoid organs, including small lymphoid organs. Light also offers the the possibility for simultaneous measurement from a wide range of spatial locations, and the ability to access specific cellular subtypes and subcellular domains.

This versatile family of genetically encoded optical tools is important for modulating integrin biology and T cell migration in a clinical setting. For example, and not to be limiting, in tumor immunology, where adoptive cell transfer has been a successful strategy for anticancer immunotherapy, the therapeutic efficiency of in vitro activated autologous T cells is dependent upon access of the T cells to the tumor sites once they are transferred to patients. A photoactivatable chemokine receptor can guide autologous T cells to the location of a tumor using non-invasive light stimulation to induce directional migration. Currently, hematopoietic stem cells are of increasing interest due to their therapeutic potential. Because transplantation protocols use intravenous injections, diseases that require hematopoietic stem cell transplantation would fail to rescue lethally irradiated recipients if the homing potential of the stem cells was impaired. Photoactivatable chemokine receptors can be important in guiding stem cell migration to the damaged tissues.

### Constructs

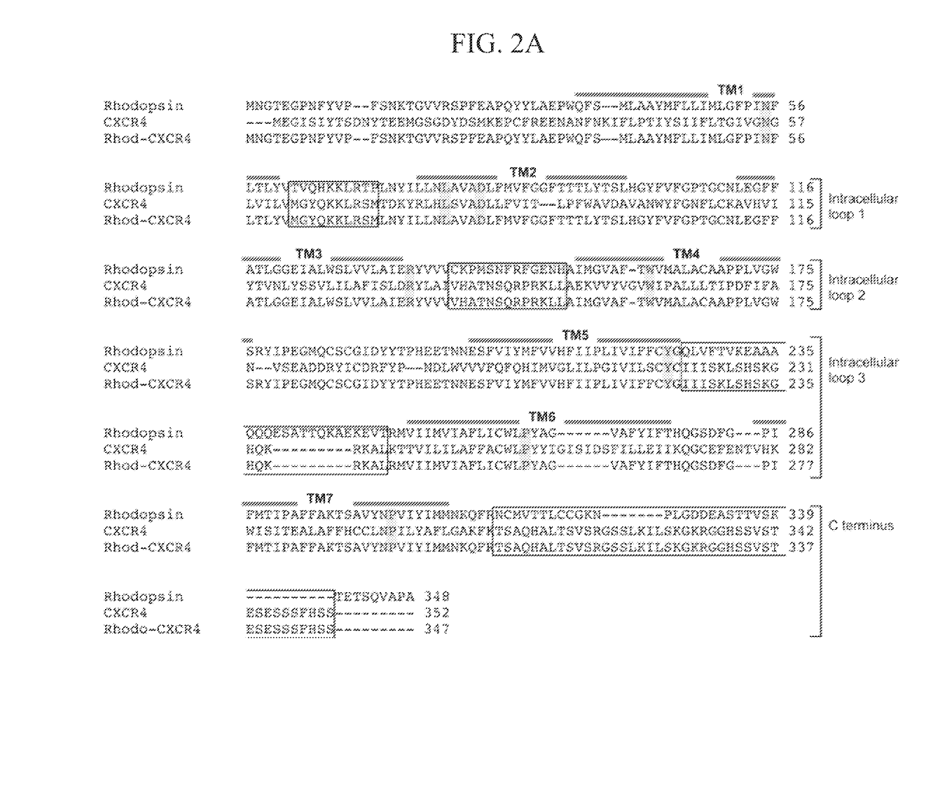

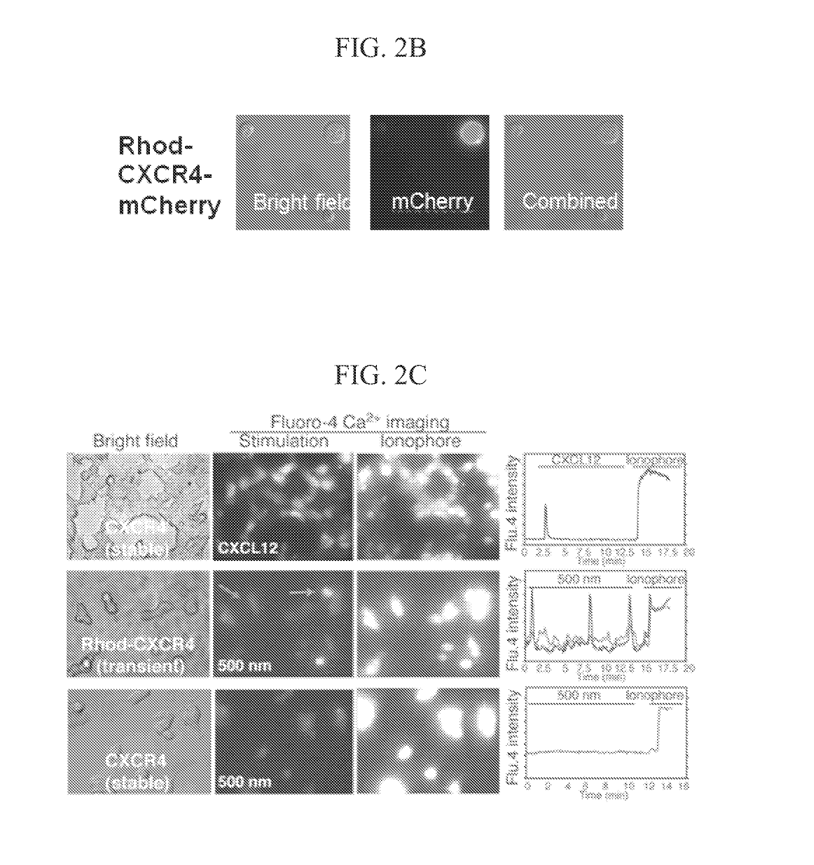

To enable optical control over intracellular signaling in mammals (FIG. 1), shared structure-function relationships among GPCRs was utilized to develop and express a rhodopsin/chemokine receptor chimera with novel transduction logic that couples signal to effector. The intracellular loops of rhodopsin were replaced with those of CXCR4 by first aligning conserved residues of the Gᵢ-coupled human CXCR4 (NCBI Accession No. NM_{—}003467) with the Gₜ-coupled bovine rhodopsin (NCBI accession no. P02699: FIG. 2A). Exchanges of intracellular regions (including carboxy-terminal domains) based on structural models (FIG. 1) were engineered to transfer G-protein coupling from Gₜ to Gᵢ and optimize expression of the chimera in mammalian cells. A nucleic acid encoding the C-terminal of the chimera (Rhod-CXCR4) fused to a fluorescent protein (mCherry) was constructed. Transient transfections of Rhod-CXCR4-mCherry in human primary T cells confirmed plasma membrane expression of the construct (FIG. 2B). Upon activation by a range of ligands, native receptors can explore multiple ensemble states to recruit canonical and non-canonical pathways in ligand-biased signaling. Photoactivatable chemokine receptors are likely to select multiple active ensemble states upon sensing light, in a manner dependent on biological context. To assess functional Rhod-CXCR4 expression, [Ca²⁺], (intracellular calcium concentration) was imaged in HEK293 cells transfected with WT CXCR4 or with Rhod-CXCR4. Fluorescence imaging of [Ca²⁺]ᵢ, demonstrated that green light stimulation (500 nm) was sufficient to drive prominent downstream [Ca²⁺], signals in cells expressing Rhod-CXCR4, but not in control cells (WT CXCR4), indicating functional expression of Rhod-CXCR4 (FIG. 2C).

### Light-Induced Chemokine Signals

To test the specificity of the long-term signaling controlled by Rhod-CXCR4, HEK293 cells are transiently transfected and illuminated with ˜500±20 nm light for 1-2 min. Cells are then lysed and analyzed for levels of phosphorylated AKT and Erk1/2 by Western blot. These levels are compared with phosphorylation levels achieved with pharmacological stimulation of the wild-type CXCR4. For further indication of the signaling specificity of the chimeric protein, studies can be performed to show that optical stimulation of cells expressing the Rhod-CXCR4 construct is unable to modulate cGMP levels (downstream signals of rhodopsin). Similar assays can be performed to confirm that Rhod-CXCR4 retains an action spectrum close to that of native rhodopsin (˜500 nm).

### Light-Mediated Directional Cell Migration

Light-mediated T cell migration is examined by showing directional migration of cells that express Rhod-CXCR4 using localized light stimulation. First, activation of lamellipodia by Rhod-CXCR4 is examined in HEK293 cells. These cells will remain quiescent when illuminated with wavelengths longer than the rhodopsin absorbance (>500 nm), but within seconds after switching to 500 nm, lamellipodial protrusions and membrane ruffles will appear around the cell edges. To show that this effect is due to Rhod-CXCR4, kymograms are used to quantify maximum protrusion length. An important advantage of Rhod-CXCR4 is its ability to precisely control the subcellular location of CXCR4 activation. Whether irradiation of 20 μm spots at the edge of HEK293 cells expressing Rhod-CXCR4 generates large protrusions clearly localized adjacent to the point of irradiation are examined. Whether movement of a laser spot to a different position leads to cessation of ruffling or protrusion at the initial irradiation position, and new activity appearing where the laser spot is brought to rest is also determined. To test directional migrations in T cells, human primary T cells are transiently transfected with Rhod-CXCR4 and placed in a cover glass heat chamber coated with ICAM-1. The ability of Rhod-CXCR4 alone to control polarized and directional migration is confirmed by repeated irradiation at the cell edge, which can be used to produce prolonged cell movement by generating consistent chemotaxis signals toward the direction of light stimulation.

### Light-Mediated Recruitment of T Cells In Vivo

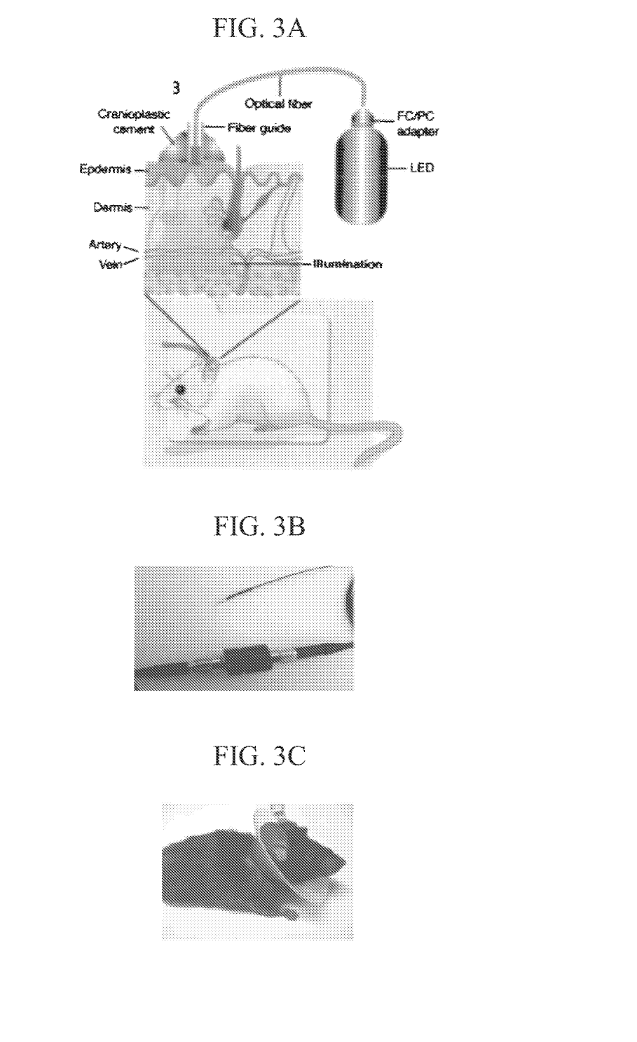

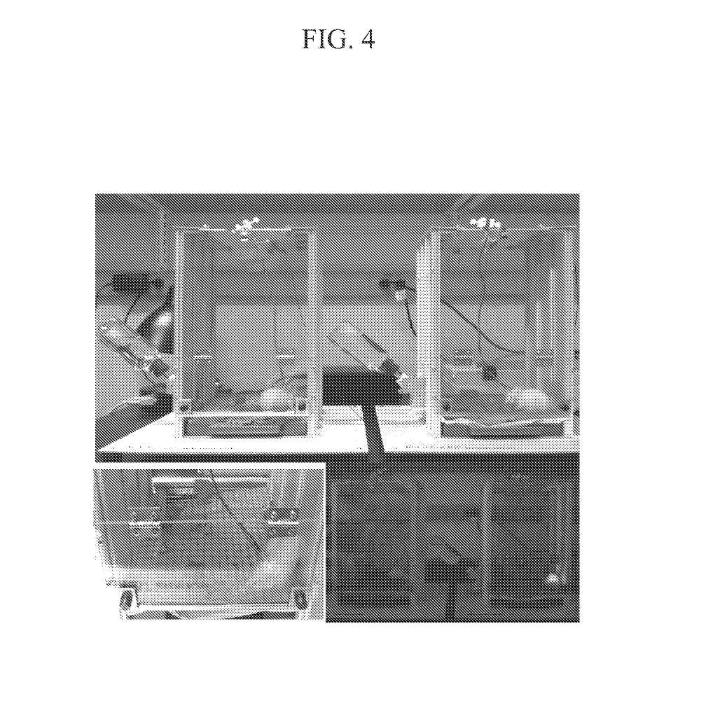

A light-mediated directional cell migration approach is used to assess the ability of precisely timed photoactivatable chemokine receptor signals to modulate in vivo T cell recruitment. In this assay, in vitro-activated T cells are transfected with Rhod-CXCR4-mCherry and then adoptively transferred into naive animals. These adoptively transferred cells are tracked by red fluorescence (mCherry), resulting in high recruitment indices with low backgrounds. For this experiment, CD4⁺ T cells are activated from T cell receptor transgenic mice on the BALB/c background that are specific for the ovalbumin peptide (D011.10 mice). The responsiveness of adoptively transferred cells to light stimulation are tested in two types of experiments. First, rapid transitions from rolling to firm adhesion are measured by locally illuminating the cremaster venule (diameter 100-200 μm) with 515 nm, 3 mm⁻² light using a confocal microscope (FluoView FV1000, Olympus). To induce directional transendothelial migration of T cells, a 515 nm laser is focused into a small circular area (diameter 2-5 μm) at the leading edge of the cell for 20-30 sec with 3% power and 10.0 ms/pixel (tornado function). For the long-term T cell recruitment assay, a thin optical fiber coupled with a cyan light-emitting diode (LED; 505 nm, Doric Lenses, Quebec, Canada) is attached on the hairless area of the unshaven mouse ear (FIGS. 3A-C). In vitro activated T cells are transfected with either GFP (green) or Rhod-CXCR4-mCherry (red), and equal numbers of green and red cells are co-transferred to WT recipient mice. The ear is harvested after 72 hr with/without light stimulation in freely moving mice (FIG. 4). Numbers of green and red cells are counted using flow cytometry and the ratio of green to red cells are measured.

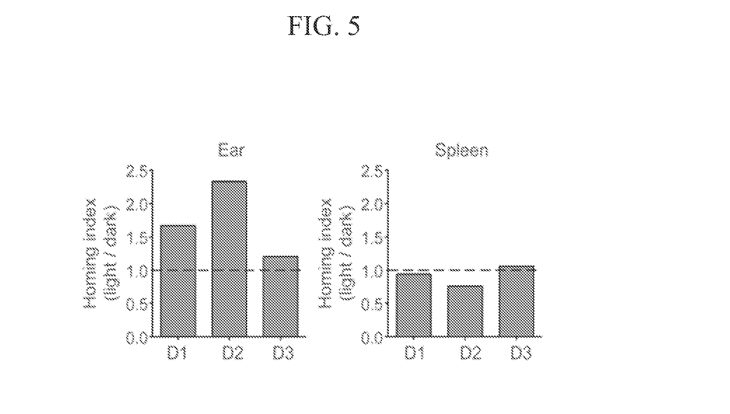

Competitive homing assays were done to assess whether CD4 T cells expressing Rhod-CXCR4 (T_{Rhod-CXCR4} cells) can effectively home to the inflamed ear in response to local light stimulation. The ratio of T_{Rhod-CXCR4} cells in light:dark inflamed ears (OVA+CFA) and spleens were assessed day 1, day 2, and day 3 posttransfer of cells into recipient mice. T_{Rhod-CXCR4} cells showed enhanced homing into light activated inflamed ear (FIG. 4), while the homing to spleen was not altered (FIG. 5). The fold change in the homing index was greater in day 1 and day 2 in the presence of light stimulation. These data show that local light stimulation can successfully recruit T cells that express Rhod-CXCR4 in live mice.

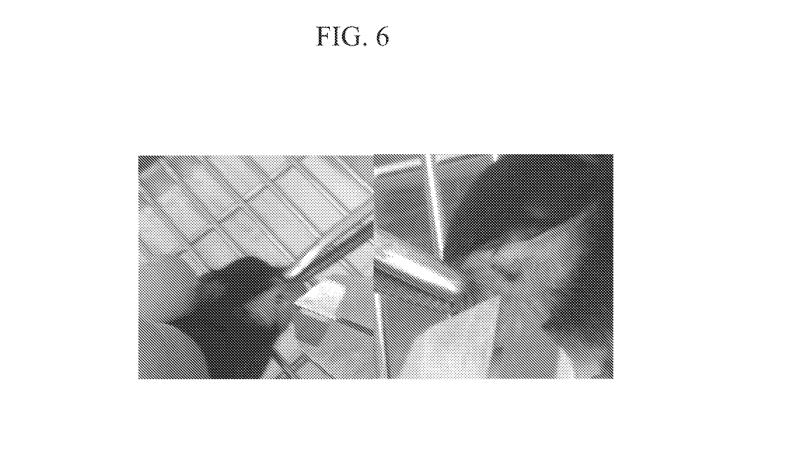

To determine if a photoactivatable chemokine receptor can guide autologous T cells to the location of a tumor using non-invasive light stimulation to induce effective tumor rejection, a B16-OVA melanoma cell line is used. To establish a mouse ear melanoma tumor, 5×10⁴ B16-OVA cells are intradermally injected into one ear pima of the recipient C57BL/6 (FIG. 6). In the meantime, CD8⁺ T cells are purified from OT-I⁺CD45.1⁺ mice and stimulated with irradiated splenocytes in 1 mM SIINFEKL OVA peptide containing media. Retrovirus infection of Rhod-CXCR4 is performed. 1×10⁶ Rhod-CXCR4⁺CD8 T cells are then transferred intravenously into the tumor-bearing recipient at day 7. Following the T cell transfer, an optical fiber is attached at the ear tumor site from day 7 to day 14. Starting at day 5, tumor growth is monitored every other day by measuring the diameter of the tumor. These measurements are used to establish growth curves.

Light-Mediated Recruitment of Stem Cells into Spinal Cord Injury

Spinal cord injury (SCI) is a devastating injury that can lead to irreversible neurological deficits. The current recommended treatments for SCI includes exogenous stem cell therapy. However, its application is limited. By stimulating migration of transplanted exogenous stem cells clinical outcomes can be improved. Bone marrow stromal cells (BMSCs) are non-hematopoietic multipotent stem cells capable of trans-differentiating into neurons, astrocytes or oligodendrocytes, BMSCs have the potential to restore injured spinal cord tissue and promote functional recovery.

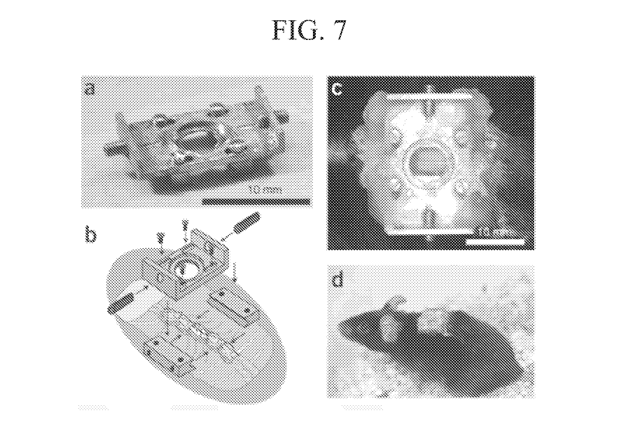

C57BL/6 mice are used in this study, SCI is induced using the modified weight-drop method. In brief, mice are anesthetized with pentobarbital (50 mg/kg intraperitoneally) and receive a laminectomy at the T10 level. After the spine is immobilized stereotactically, moderate SCI will be induced by dropping a weight of 1-3 g from a height of 2-3 cm onto an impounder (diameter, 0.2 cm) gently placed on the spinal cord (See, for example Farrar et al. “Chronic in vivo imaging in the mouse spinal cord using an implanted chamber,” Nat. Methods 22:9(3) 297-302 (2012)). Immediately after injury, Rhod-CXCR4 expressing BMSC (1×10) are injected into the mice through the tail vein, Following the cell transfer, an optical fiber is attached at the injury site through a custom-designed chamber (FIG. 7). Light stimulation is performed during the first 7 days. In order to assess restoration of injured spinal cord tissue and the extent of functional recovery neurological and histological tests are performed every 3 days for a total of 21 days.

Light-Mediated Recruitment of Regulatory T Cells into Diabetic Pancreas

Type 1 diabetes (T1D) results from the T cell-mediated destruction of insulin-producing β-cells situated in the islets of Langerhans within the pancreas, A complex interplay between genetic and environmental factors is thought to initiate disease which manifests after destruction of approximately 90% of the β-cells. Foxp3⁺ regulatory T cells (Tregs) are crucial for the maintenance of lymphoid homeostasis and self-tolerance. In the NOD mouse model, transfer of Tregs can protect from diabetes. Conversely, genetic deficiencies that reduce Treg numbers result in accelerated autoimmune diabetes.

The following animal model can be used to assess the feasibility of light-mediated recruitment of ex vivo expanded autologous polyclonal Tregs in T1D mouse to reduce diabetes severity and treat the autoimmune response underlying T1D. For this study, an NOD mouse model can be used. BDC2.5 TCR Tg mice express a TCR specific for an islet antigen expressed in the granules of β cells. Treg cells are purified from BDC2.5 and expanded using the anti-CD3/anti-CD28 plus IL-2 cocktail. The CD4⁺ CD62L⁺ CD25⁻ and T_{regs} from BDC2.5 TCR Tg mice are transfected with Rhod-CXCR4 and expanded using immobilized MHC peptide dimers. 2×10⁶ Tregs cells are transferred into NOD mice. Following the cell transfer, an optical fiber is surgically inserted into the recipient mouse. Access is gained from the splenic side and the fiber is inserted into the tail region. This leaves the vascular supply originating from the superior and inferior pancreaticoduodenal arteries intact. Light stimulation is performed during the first 7 days, and the blood glucose for each individual recipient mouse is monitored every day for a total of 21 days.

1. A chimeric photoactivatable polypeptide comprising an opsin membrane receptor, wherein an intracellular domain of the opsin membrane receptor is replaced with a corresponding intracellular domain of a chemokine receptor, a sphingosine-1-phosphate receptor or an ATP receptor.

2. The polypeptide of claim 1, wherein the opsin intracellular domain is selected from the group consisting of the first intracellular domain, the second intracellular domain, the third intracellular domain and the intracellular carboxy-terminal domain.

3. The polypeptide of claim 1, wherein two or more intracellular domains of the opsin receptor are replaced with the corresponding intracellular domains of the chemokine receptor.

4. The polypeptide of claim 1, wherein the opsin receptor is selected from the group consisting of a mammalian opsin receptor and a bacterial opsin receptor.

5. The polypeptide of claim 4, wherein the opsin receptor is rhodopsin.

6. The polypeptide of claim 1, wherein the chemokine receptor is selected from the group consisting of CXCR4, CXCR7, CXCR1, CXCR2, CXCR3, CXCR5, CXCR6, CCR1, CCR2, CCR3, CCR4, CCR5, CCR6, CCR7, CCR8, CCR9, CCR10, CCR11, XCR1 and CS3CR1.

7. The polypeptide of claim 1, wherein the sphingosine-1-phosphate receptor is selected from the group consisting of S1P1, S1P2 and S1P3.

8. The polypeptide of claim 1, wherein the ATP receptor is a P2y receptor

9. The polypeptide of claim 6, wherein the chemokine receptor is CXCR4.

10. The polypeptide of claim 1, wherein the opsin membrane receptor is a mammalian rhodopsin and the chemokine receptor is CXCR4.

11. The polypeptide of claim 10, wherein the polypeptide comprises a polypeptide sequence comprising SEQ ID NO: 1.

12. A nucleic acid sequence encoding the polypeptide of claim 1.

13. A vector comprising the nucleic acid of claim 12.

14. A host cell comprising the vector of claim 13.

15. The host cell of claim 14, wherein the cell is a T cell, a B cell, a stem cell, an NK cell, a macrophage, a neutrophil, an eosinophil, a monocyte, a dendrite cell, an endothelial cell, and a keratinocyte.

16. An animal comprising the host cell of claim 14.

17. A transgenic non-human animal, wherein the genome of the animal comprises the nucleic acid of claim 12, operably linked to a cell-specific or tissue specific promoter.

18. A method of inducing cell migration comprising exposing a cell that expresses the polypeptide of claim 1 to a visible light source.

19. The method of claim 18, wherein the visible light source is a laser or a light emitting diode.

20. The method of claim 19, wherein the visible light source emits light at a wavelength of about 450 to 515 nm.

21. The method of claim 18, wherein the cell is in vitro, ex vivo, or in vivo.

22. A method of treating cancer in a subject comprising administering to the subject a cell that expresses the polypeptide of claim 1 and exposing the cell in the subject to a visible light source, wherein the subject has cancer.

23. The method of claim 22, wherein the cancer is selected from the group consisting of skin cancer, colon cancer, breast cancer, prostate cancer, esophageal cancer, rectal cancer, throat cancer, lung cancer, stomach cancer.

24. The method of claim 22, wherein the cell is removed from the subject and transfected ex vivo with a nucleic acid encoding the polypeptide, prior to administering the cell to the subject.

25. The method of claim 22, wherein the cell is administered to the subject at a surgical site.

26. The method of claim 22, wherein the cell is a T cell, a stem cell or an NK cell.

27. A method of treating a spinal cord injury comprising transplanting a stem cell that expresses the polypeptide of claim 1 into the spinal cord of a subject and exposing the cell in the subject to a visible light source, wherein the subject has a spinal cord injury.

28. A method of treating an autoimmune disorder or preventing transplant rejection in a subject by administering a regulatory T cell that expresses the polypeptide of claim 1 to the subject and exposing the cell in the subject to a visible light source, wherein the subject has an autoimmune disorder or has received an organ transplant.

29. A method of treating an infection in a subject by administering a regulatory T cell that expresses the polypeptide of claim 1 to the subject and exposing the cell in the subject to a visible light source, wherein the subject has an infection.

30. The method of claim 18, wherein the visible light source is a laser or a light emitting diode.

31. The method of claim 30, wherein the visible light source emits light at a wavelength of about 450 to 515 nm.
